(19) United States
(12) Patent Application Publication
(10) Pub. No.: US 2026/0256672 A1
(43) Pub. Date: Sep. 3, 2026

(54) COMPOSITION COMPRISING SKIN CARE ACTIVE INGREDIENT OR CELLULOSE COMPOUND

(71) Applicant: L'OREAL, PARIS (FR)

(72) Inventors: Romain TACHON, Kawasaki-shi (JP); Mina MUTO, Kawasaki-shi (JP); Yusuke IIMA, Kawasaki-shi (JP); Nahoko NAKASHIMA, Kawasaki-shi (JP)

(21) Appl. No.: 18/881,602

(22) PCT Filed: Jun. 23, 2023
(86) PCT No.: PCT/JP2023/024299

§ 371 Date: Jan. 6, 2025

(30) Foreign Application Priority Data
Jul. 12, 2022 (JP) 2022-111767
Jul. 12, 2022 (JP) 2022-111768
Aug. 5, 2022 (FR) 2208131
Aug. 18, 2022 (FR) 2208355

(51) Int. Cl. A61K 8/73; A61K 8/22; A61K 8/34; A61K 8/368; A61K 8/41; A61K 8/42; A61K 8/60; A61K 8/67; A61K 8/81; A61K 8/99; A61Q 19/02; A61Q 19/08

(52) U.S. Cl. CPC A61K 8/731; A61K 8/22; A61K 8/345; A61K 8/347; A61K 8/368; A61K 8/41; A61K 8/42; A61K 8/60; A61K 8/675; A61K 8/676; A61K 8/737; A61K 8/8152; A61K 8/99; A61Q 19/02; A61Q 19/08

(57) ABSTRACT

A composition contains: (a) at least one oil; (b) at least one diol having at least one carbon chain containing 3 or more carbon atoms; (c) (c1) at least one skin care active ingredient or (c2) at least one cellulose compound; and (d) water. A composition can enhance or improve the penetration of the ingredient (c1) into the skin as compared to a case in which the ingredient (b) is not used. A composition can enhance glow of the skin, as compared to a case in which the ingredient (c2) is not used, while providing fresh texture when being used.

### TECHNICAL FIELD

The present invention relates to a composition, preferably a cosmetic composition, and more preferably a skin cosmetic composition, which comprises at least one skin care active ingredient or at least one cellulose compound.

### BACKGROUND ART

First, there have been a wide variety of skin care active ingredients used in the field of cosmetics and dermatological compositions.

It is preferable for skin care active ingredients to be absorbed by the skin or penetrated into the skin as much as possible. However, skin absorption or skin penetration of such skin care active ingredients is not easy due to the barrier function of the skin. Thus, one possible approach to enhance the skin penetration of a skin care active ingredient may be to use a skin penetration enhancer with the skin care active ingredient.

For example, WO 2018/097303 uses a combination of a single polyglyceryl fatty acid ester and niacinamide in a nano- or micro-emulsion including a ceramide in order to enhance the skin penetration of the ceramide.

Second, glowy skin is one of important consumers' needs seen in many countries such as China, Japan and the U.S.

Oil-based cosmetic products have been used to enhance glow of the skin. However, there is a technical challenge that oil-based cosmetic products often provide unpleasant feeling to touch such as greasy texture when being used. On the other hand, in general, water-based cosmetic products can provide not greasy but fresh texture. However, water-based cosmetic products are difficult to enhance glow of the skin.

JP-B-3774180 discloses (cf. the Examples section thereof) some specific dermatological compositions including an N-acyl sarcosine alkyl ester, a specific diol, and 12-hydroxystearic acid. However, JP-B-3774180 does not teach the use of celluloses or cellulose derivatives in order to enhance glow of the skin.

JP-A-2004-75639 also discloses (cf. the Examples section thereof) some specific dermatological compositions including a viscous oil, an N-acyl sarcosine alkyl ester, and a specific diol. However, JP-A-2004-75639 does not teach the use of celluloses or cellulose derivatives in order to enhance glow of the skin.

### DISCLOSURE OF INVENTION

There has been a need for a composition including a skin care active ingredient, which can enhance or improve the penetration of the skin care active ingredient into the skin, based on a new skin penetration enhancer.

Also, there has been another need for a composition including oil and can enhance glow of skin, while providing fresh texture when being used.

A first objective of the present invention is to provide a composition which can enhance or improve the penetration of a skin care active ingredient into the skin.

A second objective of the present invention is to provide a composition which comprises oil and can enhance glow of the skin, while providing fresh texture when being used.

The above first or second objective of the present invention can be achieved with a composition comprising:
(a) at least one oil;
(b) at least one diol having at least one carbon chain comprising 3 or more carbon atoms;
(c) (c1) at least one skin care active ingredient or (c2) at least one cellulose compound; and
(d) water.

The (a) oil may be selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, provided that
the (i) fatty alcohols and the (ii) fatty acids comprise a branched alkyl group or a linear or branched alkenyl group, each having 4 to 30, preferably 5 to 26, and more preferably 6 to 22 carbon atoms,
the (iii) monovalent carboxylic acid monoesters comprise a branched alkyl group or a linear or branched alkenyl group, each having 4 to 30, preferably 5 to 26, and more preferably 6 to 22 carbon atoms,
the (iv) divalent carboxylic acid diesters comprise at least one branched alkyl group having 3 to 10, preferably 3 to 8, and more preferably 3 to 6 carbon atoms, and an alkylene group having 6 to 18, preferably 6 to 14, and more preferably 6 to 10 carbon atoms,
the (v) N-acyl amino acid esters comprise a branched alkyl group having 3 to 10, preferably 3 to 8, and more preferably 3 to 6 carbon atoms, and a linear or branched acyl group having 4 to 30, preferably 5 to 26, and more preferably 6 to 22 carbon atoms, and
the (vi) alkyl lactates comprise a linear or branched alkyl group or a linear or branched alkenyl group, each having 4 to 30, preferably 5 to 26, and more preferably 6 to 22 carbon atoms.

The (a) oil may be selected from (v) N-acyl amino acid esters comprising, at least, a branched alkyl group having 3 to 10, preferably 3 to 8, and more preferably 3 to 6 carbon atoms, preferably (v) N-acyl amino acid esters represented by the following general formula (5):

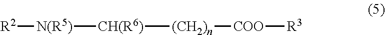

wherein
R² represents a linear or branched, acyl group, having 4 to 30, preferably 5 to 26, and more preferably 6 to 22 carbon atoms,
R³ represents a branched alkyl group having 3 to 10, preferably 3 to 8, and more preferably 3 to 6 carbon atoms,
R⁵ represents a hydrogen atom, or a linear or branched alkyl group having 1 to 4 carbon atoms,
R⁶ represents a hydrogen atom, or a linear or branched alkyl group having 1 to 4 carbon atoms which may be substituted with a hydroxy group or a phenyl group, and
n represents an integer from 0 to 2,
and more preferably the (a) oil is isopropyl lauroyl sarcosinate.

The amount of the (a) oil(s) in the composition according to the present invention may range from 0.5% to 25% by weight, preferably from 1% to 20% by weight, and more preferably from 2% to 15% by weight, relative to the total weight of the composition.

The (b) diol having at least one carbon chain comprising 3 or more carbon atoms may be selected from the group consisting of propylene glycol, dipropylene glycol, polypropylene glycol, butylene glycol, dibutylene glycol, polybutylene glycol, pentylene glycol, dipentylene glycol, hexylene glycol, dihexylene glycol, and a mixture thereof.

The weight ratio of the amount of the (b) diol having at least one carbon chain comprising 3 or more carbon atoms to the amount of the (a) oil may be 4 or more, preferably 5 or more, and more preferably 6 or more.

The (c1) skin care active ingredient may have a Log P value of 4 or less, preferably from −4.5 to 4.0, and more preferably from −4.0 to 3.5.

The (c1) skin care active ingredient may be a skin care cosmetic active ingredient, preferably a skin whitening or anti-wrinkle ingredient, and more preferably selected from the group consisting of ascorbyl glucoside, 3-O-ethyl ascorbic acid, tranexamic acid, hydroxypropyl tetrahydropyrantriol, niacinamide, salicylic acid, phenylethyl resorcinol, and a mixture thereof.

The amount of the (c1) skin care active ingredient(s) in the composition according to the present invention may range from 0.01% to 25% by weight, preferably from 0.05% to 20% by weight, and more preferably from 0.1% to 15% by weight, relative to the total weight of the composition.

The (c2) cellulose compound may be selected from the group consisting of unmodified cellulose and nonionic or anionic cellulose ethers.

The (c2) cellulose compound may be selected from the group consisting of cellulose, cellulose gum, hydroxymethylcellulose, hydroxyethylcellulose, hydroxypropylmethylcellulose, sodium carboxymethylcellulose, and a mixture thereof.

The amount of the (c2) cellulose compound(s) in the composition according to the present invention may range from 0.01% to 40% by weight, preferably from 0.05% to 35% by weight, and more preferably from 0.1% to 30% by weight, relative to the total weight of the composition.

The composition according to the present invention may have a turbidity of 120 NTU or less, preferably 90 NTU or less and more preferably 60 NTU or less.

The composition according to the present invention may be a cosmetic composition, preferably a skin cosmetic composition, and more preferably a skin care cosmetic composition.

The present invention also relates to a cosmetic process for treating a keratin substance, preferably skin, comprising the step of applying the composition according to the present invention.

### BEST MODE FOR CARRYING OUT THE INVENTION

After diligent research, the inventors have discovered that it is possible to provide a composition which can enhance or improve the penetration of a skin care active ingredient into the skin by using a new skin penetration enhancer, and that it is possible to provide a composition which comprises oil and can enhance glow of the skin, while providing fresh texture when being used.

Thus, the present invention relates to a composition comprising:
(a) at least one oil;
(b) at least one diol having at least one carbon chain comprising 3 or more carbon atoms;
(c) (c1) at least one skin care active ingredient or (c2) at least one cellulose compound; and
(d) water.

If the composition according to the present invention comprises (c1) at least one skin care active ingredient, the composition according to the present invention can enhance or improve the penetration of the (c1) skin care active ingredient into the skin, as compared to a case in which the ingredient (b) is not used.

The (b) at least one diol having at least one carbon chain comprising 3 or more carbon atoms can function as a skin penetration enhancer for the (c1) skin care active ingredient.

The composition according to the present invention comprises at least one oil. Accordingly, the composition according to the present invention can enhance glow of the skin, when being applied onto the skin.

If the composition according to the present invention comprises (c2) at least one cellulose compound, the composition according to the present invention can further enhance glow of the skin, as compared to a case in which the ingredient (c2) is not used, while providing fresh texture when being used.

The (c2) at least one cellulose compound can function as a skin glow enhancer.

The composition according to the present invention can include a relatively large amount of oil. Therefore, the composition according to the present invention can provide better cosmetic effects derived from the relatively larger amount of oil, such as a further enhanced glow of the skin.

The composition according to the present invention can also provide fresh texture when being used. Thus, the composition according to the present invention can provide less greasy texture.

The composition according to the present invention can be stable over time, even under temperature changes, e.g., changes from hot to cool temperatures and vice versa, such that they do not cause any phase separation, while maintaining uniform appearance.

The composition according to the present invention can be transparent or slightly translucent, but preferably transparent.

The composition according to the present invention does not need to include any surfactant. Thus, the amount of surfactant(s) in the composition can be limited, such as 1% by weight or less, preferably 0.1% by weight or less, and more preferably 0.01% by weight or less, relative to the total weight of the composition. It is most preferable that the composition according to the present invention comprises no surfactant.

It has been well known that surfactants can provide greasy texture and stimulation to skin.

Thus, if the composition according to the present invention includes only a limited amount of surfactant(s), the composition according to the present invention can provide even less greasy texture.

In addition, if the composition according to the present invention includes only a limited amount of surfactant(s), the composition according to the present invention can provide less stimulation to skin.

Hereinafter, the composition according to the present invention will be explained in a more detailed manner.

### [Oil]

The composition according to the present invention may comprise (a) at least one oil. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The (a) oil may be a non-polar oil such as a hydrocarbon oil, a silicone oil, or the like; a polar oil such as a plant or animal oil and an ester oil or an ether oil; or a mixture thereof.

The (a) oil may be selected from the group consisting of oils of plant or animal origin, synthetic oils, silicone oils, hydrocarbon oils, and fatty alcohols.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

Esters of C₄-C₂₂ dicarboxylic or tricarboxylic acids and of C₁-C₂₂ alcohols, and esters of monocarboxylic, dicarboxylic, or tricarboxylic acids and of non-sugar C₄-C₂₆ dihydroxy, trihydroxy, tetrahydroxy, or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides, or polysaccharides.

Examples of suitable sugars that may be mentioned include sucrose (or saccharose), glucose, galactose, ribose, fucose, maltose, fructose, mannose, arabinose, xylose, and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, linoleates, linolenates, caprates, and arachidonates, or mixtures thereof such as, especially, oleopalmitate, oleostearate, and palmitostearate mixed esters, as well as pentaerythrityl tetraethyl hexanoate.

More particularly, use is made of monoesters and diesters and especially sucrose, glucose, or methylglucose monooleates or dioleates, stearates, behenates, oleopalmitates, linoleates, linolenates, and oleostearates.

An example that may be mentioned is the product sold under the name Glucate® DO by the company Amerchol which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri (2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri (caprate/caprylate), and glyceryl tri (caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of the formula:
   with

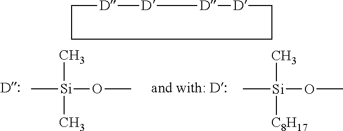

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

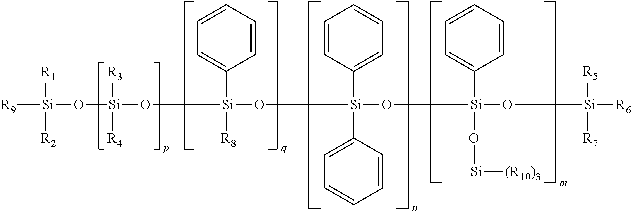

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl, or butyl radicals, and
m, n, p, and q are, independently of each other, integers from 0 to 900 inclusive, preferably 0 to 500 inclusive, and more preferably 0 to 100 inclusive,
with the proviso that the sum, n+m+q, is an integer other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250, and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

The hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane, and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from cetyl alcohol, octyldodecanol, hexyldecanol, and mixtures thereof.

It is also preferable that the (a) oil be chosen from oils with a molecular weight below 600 g/mol.

Preferably, the (a) oil has a low molecular weight such as below 600 g/mol, chosen among ester oils with a short hydrocarbon chain or chains (C₁-C₁₂) (e.g., isopropyl lauroyl sarcosinate, isopropyl myristate, isopropyl palmitate, isononyl isononanoate, and ethyl hexyl palmitate), silicone oils (e.g., volatile silicones such as cyclohexasiloxane), hydrocarbon oils (e.g., isododecane, isohexadecane, and squalane), branched and/or unsaturated fatty alcohol (C₁₂-C₃₀) type oils such as octyldodecanol and oleyl alcohol, and ether oils such as dicaprylyl ether.

Hereafter, preferred embodiments of the (a) oil are described.

It is preferable that the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, under the following conditions:
the (i) fatty alcohols and the (ii) fatty acids comprise a branched alkyl group or a linear or branched alkenyl group, each having 4 to 30, preferably 5 to 26, and more preferably 6 to 22 carbon atoms,
the (iii) monovalent carboxylic acid monoesters comprise a branched alkyl group or a linear or branched alkenyl group, each having 4 to 30, preferably 5 to 26, and more preferably 6 to 22 carbon atoms,
the (iv) divalent carboxylic acid diesters comprise at least one, preferably two, branched alkyl group having 3 to 10, preferably 3 to 8, and more preferably 3 to 6 carbon atoms, and an alkylene group having 6 to 18, preferably 6 to 14, and more preferably 6 to 10 carbon atoms,
the (v) N-acyl amino acid esters comprise a branched alkyl group having 3 to 10, preferably 3 to 8, and more preferably 3 to 6 carbon atoms, and a linear or branched acyl group having 4 to 30, preferably 5 to 26, and more preferably 6 to 22 carbon atoms, and
the (vi) alkyl lactates comprise a linear or branched alkyl group or a linear or branched alkenyl group, each having 4 to 30, preferably 5 to 26, and more preferably 6 to 22 carbon atoms.

In the present specification, the “alkyl group” refers to a linear or branched monovalent saturated aliphatic hydrocarbon group.

As the linear alkyl group having 4 to 30 carbon atoms, mention may be made of, for example, an n-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, and an n-eicosyl group.

As the branched alkyl group having 4 to 30 carbon atoms, mention may be made of, for example, an isobutyl group, a sec-butyl group, a tert-butyl group, an isopentyl group, a neopentyl group, an isohexyl group, an neohexyl group, an isoheptyl group, a neoheptyl group, an isooctyl group, an isononyl group, an isodecyl group, an isoundecyl group, an isododecyl group, an isotridecyl group, an isotetradecyl group, an isopentadecyl group, an isohexadecyl group, an isoheptadecyl group, an isooctadecyl group, an isononadecyl group, and an isoeicosyl group.

As the branched alkyl group having 3 to 10 carbon atoms, mention may be made of, for example, an isopropyl group, an isobutyl group, a sec-butyl group, a tert-butyl group, an isopentyl group, a neopentyl group, an isohexyl group, an neohexyl group, an isoheptyl group, a neoheptyl group, an isooctyl group, an isononyl group, and an isodecyl group.

In the present specification, the “alkenyl group” refers to a linear or branched monovalent unsaturated aliphatic hydrocarbon group having at least one carbon-carbon double bond.

As the linear alkenyl group having 4 to 30 carbon atoms, mention may be made of, for example, a butenyl group, a pentenyl group, a hexenyl group, a heptenyl group, a heptadienyl group, a heptatrienyl group, an octenyl group, an octadienyl group, an octatrienyl group, a nonenyl group, a nonadienyl group, a nonatrienyl group, a decenyl group, a decadienyl group, a decatrienyl group, an undecenyl group, an undecadienyl group, an undecatrienyl group, a dodecenyl group, a dodecadienyl group, a dodecatrienyl group, a tridecenyl group, a tridecadienyl group, a tridecatrienyl group, a tetradecenyl group, a tetradecadienyl group, a tetradecadienyl group, a tetradecatrienyl group, a pentadecenyl group, a pentadecadienyl group, a pentadecatrienyl group, a hexadecenyl group, a hexadecadienyl group, a hexadecatrienyl group, a heptadecenyl group, a heptadecadienyl group, a heptadecatrienyl group, an octadecenyl group, an octadecadienyl group, an octadecatrienyl group, a nonadecenyl group, a nonadecadienyl group, a nonadecatrienyl group and an eicosenyl group.

As the branched alkenyl group having 4 to 30 carbon atoms, mention may be made of, for example, an isobutenyl group, a sec-butenyl group, an isopentenyl group, an isohexenyl group, an isoheptenyl group, an isooctenyl group, an isononenyl group, an isodecenyl group, an isodecadienyl group, an isodecatrienyl group, an isoundecenyl group, an isoundecadienyl group, an isoundecatrienyl group, an isododecenyl group, an isododecadienyl group, an isododecatrienyl group, an isotridecenyl group, an isotridecadienyl group, an isotridecatrienyl group, an isotetradecenyl group, an isotetradecadienyl group, an isotetradecadienyl group, an isotetradecatrienyl group, an isopentadecenyl group, an isopentadecadienyl group, an isopentadecatrienyl group, an isohexadecenyl group, an isohexadecadienyl group, an isohexadecatrienyl group, an isoheptadecenyl group, an isoheptadecadienyl group, an isoheptadecatrienyl group, an isooctadecenyl group, an isooctadecadienyl group, an isooctadecatrienyl group, an isononadecenyl group, an isononadecadienyl group, an isononadecatrienyl group and an isoeicosenyl group.

In the present specification, the “acyl group” refers to R—CO— group wherein R denotes a linear or branched monovalent saturated aliphatic hydrocarbon group (alkyl group).

As the linear or branched acyl group having 4 to 30 carbon atoms, mention may be made of, for example, a butanoyl group, an iso-butanoyl group, a sec-butanoyl group, a tert-butanoyl group, an n-pentanoyl group, an isopentanoyl group, an neopentanoyl group, an n-hexanoyl group, an isohexanoyl group, a neohexanoyl group, an n-heptanoyl group, an isoheptanoyl group, a neoheptanoyl group, an n-octanoyl group, an isooctanoyl group, an n-nonanoyl group, an isononanoyl group, an n-decanoyl group, an isodecanoyl group, an n-undecanoyl group, an isoundecanoyl group, an n-dodecanoyl group (lauroyl group), an isododecanoyl group, an n-tridecanoyl group, an isotridodecanoyl group, an n-tetradecanoyl group (myristoyl group), an isotetradecanoyl group, an n-pentadecanoyl group, an isopentadecanoyl group, an n-hexadecanoyl group (palmitoyl group), an isohexadecanoyl group, an n-heptadecanoyl group, an isohexadecanoyl group, an n-octadecanoyl group (stearoyl group), an isooctadecanoyl group, an n-nonadecanoyl group, an isononadecanoyl group, an n-eicosanoyl group, and an isoeicosanoyl group.

The (i) fatty alcohol may be represented by the following general formula (1):

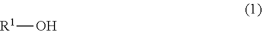

wherein
R¹ represents a branched alkyl group, or a linear or branched alkenyl group, each having 4 to 30, preferably 5 to 26, and more preferably 6 to 22 carbon atoms.

As examples of the branched alkyl group, and the linear or branched alkenyl group, each having 4 to 30 carbon atoms, mention may be made of those listed above.

As the (i) fatty alcohol represented by the formula (1), mention may be made of, for example, 2-ethylhexanol, palmitoleyl alcohol, isostearyl alcohol, elaidyl alcohol, oleyl alcohol, linoleyl alcohol, ricinoleyl alcohol, erucyl alcohol, and a mixture thereof.

It is preferable that the (i) fatty alcohol be selected from the group consisting of isostearyl alcohol, oleyl alcohol, and a mixture thereof.

The (ii) fatty acid may be represented by the following general formula (2):

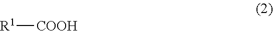

wherein
R¹ represents a branched alkyl group, or a linear or branched alkenyl group, each having 4 to 30, preferably 5 to 26, and more preferably 6 to 22 carbon atoms.

As examples of the branched alkyl group, and the linear or branched alkenyl group, each having 4 to 30 carbon atoms, mention may be made of those listed above.

As the (ii) fatty acid represented by the formula (2), mention may be made of, for example, isocaproic acid, isononanoic acid, crotonic acid, isolauric acid, myristoleic acid, isopalmitic acid, palmitoleic acid, isostearic acid, elaidic acid, oleic acid, linoleic acid, linolenic acid, arachidonic acid, eicosapentaenoic acid, tetracosapentaenoic acid, docosahexaenoic acid, nervonic acid, and a mixture thereof.

It is preferable that the (ii) fatty acid be selected from the group consisting of isostearic acid, oleic acid, and a mixture thereof.

The (iii) monovalent carboxylic acid monoester may be represented by the following general formula (3):

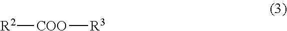

wherein
R² represents a branched alkyl group or a linear or branched alkenyl group, each having 4 to 30, preferably 5 to 26, and more preferably 6 to 22 carbon atoms, and
R³ represents a branched alkyl group having 3 to 10, preferably 3 to 8, and more preferably 3 to 6 carbon atoms.

As examples of the branched alkyl group and the linear or branched alkenyl group, each having 4 to 30 carbon atoms, mention may be made of those listed above.

As examples of the branched alkyl group having 3 to 10 carbon atoms, mention may be made of those listed above.

As the (iii) monovalent carboxylic acid monoester represented by the formula (3), mention may be made of, for example, isopropyl palmitate, isopropyl myristate, isocetyl stearate, 2-ethylhexyl isononanoate, isononyl isononanoate, isodecyl neopentanoate, isostearyl neopentanoate, and a mixture thereof.

It is preferable that the (iii) monovalent carboxylic acid monoester be isopropyl isostearate.

The (iv) divalent carboxylic acid diester may be represented by the following general formula (4):

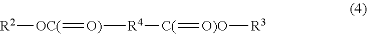

wherein
R² and R³ independently represent a branched alkyl group having 3 to 10, preferably 3 to 8, and more preferably 3 to 6 carbon atoms, and
R⁴ represents a linear or branched alkylene group having 6 to 18, preferably 6 to 14, and more preferably 6 to 10 carbon atoms.

As examples of the branched alkyl group having 3 to 10 carbon atoms, mention may be made of those listed above.

As examples of the linear or branched alkylene group having 2 to 10 carbon atoms, mention may be made of, for example, an ethylene group, a propylene group, a butylene group, a pentylene group, a hexylene group, a heptylene group, an octylene group, a nonylene group, and a decanylene group.

It is preferable that the (iv) divalent carboxylic acid diester be selected from the group consisting of diisopropyl adipate, diisopropyl sebacate, and a mixture thereof.

The (v) N-acyl amino acid ester may be represented by the following general formula (5):

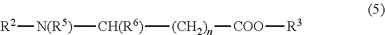

wherein
R² represents a linear or branched acyl group having 4 to 30, preferably 5 to 26, and more preferably 6 to 22 carbon atoms,
R³ represents a branched alkyl group having 3 to 10, preferably 3 to 8, and more preferably 3 to 6 carbon atoms,
R⁵ represents a hydrogen atom, or a linear or branched alkyl group having 1 to 4 carbon atoms,
Re represents a hydrogen atom, or a linear or branched alkyl group having 1 to 4 carbon atoms which may be substituted with a hydroxy group or a phenyl group, and
n represents an integer from 0 to 2.

As examples of the linear or branched acyl group having 4 to 30 carbon atoms, mention may be made of those listed above.

As examples of the branched alkyl group having 3 to 10 carbon atoms, mention may be made of those listed above.

As examples of a linear or branched alkyl group having 1 to 4 carbon atoms, mention may be made of a methyl group, an ethyl group, a propyl group, an isopropyl group, a n-butyl group, an iso-butyl group, a sec-butyl group, and a tert-butyl group.

As the (v) N-acyl amino acid ester represented by the formula (5), mention may be made of, for example, N-capryloyl sarcosine isopropyl ester, N-caprynoyl sarcosine isopropyl ester, N-lauroyl sarcosine isopropyl ester, N-coconut oil fatty acid acyl sarcosine isopropyl ester (N-cocoyl sarcosine isopropyl ester), N-myristoyl sarcosine isopropyl ester, N-palmitoyl sarcosine isopropyl ester, N-stearoyl sarcosine isopropyl ester, N-capryloyl sarcosine isobutyl ester, N-caprynoyl sarcosine isobutyl ester, N-lauroyl sarcosine isobutyl ester, N-coconut oil fatty acid acyl sarcosine isobutyl ester (N-cocoyl sarcosine isobutyl ester), N-myristoyl sarcosine isobutyl ester, N-palmitoyl sarcosine isobutyl ester, N-stearoyl sarcosine isobutyl ester, N-capryloyl glycine isopropyl ester, N-caprynoyl glysine isopropyl ester, N-lauroyl glycine isopropyl ester, N-coconut oil fatty acid acyl glycine isopropyl ester (N-cocoyl glycine isopropyl ester), N-myristoyl glycine isopropyl ester, N-palmitoyl glycine isopropyl ester, N-stearoyl glycine isopropyl ester, N-capryloyl alanine isopropyl ester, N-caprinoyl alanine isopropyl ester, N-lauroyl alanine isopropyl ester, N-coconut oil fatty acid acyl alanine isopropyl ester (N-cocoyl alanine isopropyl ester), N-myristoyl alanine isopropyl ester, N-palmitoylalanine isopropyl ester, N-stearoylalanine isopropyl ester, N-lauroyl-β-alanine isopropyl ester, N-lauroyl-N-methyl-β-alanine isopropyl ester, N-coconut oil fatty acid acyl-N-methyl-B-alanine isopropyl ester (N-cocoyl-N-methyl-β-alanine isopropyl ester) and the like.

It is preferable that the (v) N-acyl amino acid ester be isopropyl lauroyl sarcosinate. As isopropyl lauroyl sarcosine, a commercial product such as Eldew® SL-205 by Ajinomoto may be used.

The (vi) alkyl lactate may be represented by the following general formula (6):

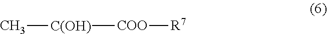

wherein
R⁷ represents a linear or branched alkyl group or a linear or branched alkenyl group, each having 4 to 30, preferably 5 to 26, and more preferably 6 to 22 carbon atoms.

As examples of the linear or branched alkyl group or the linear or branched alkenyl group, each having 4 to 30 carbon atoms, mention may be made of those listed above.

As the examples of the (vi) alkyl lactate represented by the formula (6), mention may be made of, for example, capryl lactate, caprylyl lactate, lauryl lactate, myristyl lactate, palmityl lactate, stearyl lactate, isostearyl lactate, oleyl lactate, and linoleyl lactate.

It is preferable that the (vi) alkyl lactate be selected from the group consisting of lauryl lactate, isostearyl lactate, and a mixture thereof.

The amount of the (a) oil(s) in the composition according to the present invention may be 0.5% by weight or more, preferably 1% by weight or more, and more preferably 2% by weight or more, relative to the total weight of the composition.

On the other hand, the amount of the (a) oil(s) in the composition according to the present invention may be 25% by weight or less, preferably 20% by weight or less, and more preferably 15% by weight or less, relative to the total weight of the composition.

Thus, the amount of the (a) oil(s) in the composition according to the present invention may be from 0.5% to 25% by weight, preferably from 1% to 20% by weight, and more preferably from 2% to 15% by weight, relative to the total weight of the composition.

### [Diol]

The composition according to the present invention comprises at least one (b) diol(s) having at least one carbon chain comprising 3 or more carbon atoms. Two or more (b) diol(s) having at least one carbon chain comprising 3 or more carbon atoms may be used.

The term “diol” here means a compound which has two alcohol functions. In other words, diol is an alcohol having two hydroxy groups.

The number of carbon atoms in the carbon chain may be 10 or less, preferably 8 or less, and more preferably 6 or less. It may be even more preferable that the number of carbon atoms in the carbon chain be 5 or 6.

The carbon chain comprising 3 or more carbon atoms in the ingredient (b) may be straight or branched. The carbon chain may not be interrupted by a heteroatom such as an oxygen atom, a sulfur atom and a nitrogen atom. Thus, the carbon chain may comprise consecutive carbon atoms.

It may be preferable that the number of carbon chain comprising 3 or more carbon atoms in the ingredient (b) be 1 or 2.

The total number of carbon atoms in the ingredient (b) may be 5 or more. The total number of carbon atoms in the ingredient (b) may be 10 or less, preferably 8 or less, and more preferably 6 or less. It may be even more preferable that the total number of carbon atoms in the ingredient (b) be 5 or 6.

The (b) diol(s) having at least one carbon chain comprising 3 or more (preferably 8 or less, and more preferably 6 or less) carbon atoms may have a divalent straight or branched hydrocarbon group. The divalent straight or branched hydrocarbon group which may be present in the ingredient (b) may be saturated or unsaturated. It is preferable that the (b) diol(s) having at least one carbon chain comprising 3 or more carbon atoms comprise a divalent straight or branched saturated hydrocarbon group, and more preferably a straight or branched alkylene group, such as a propylene group, a butylene group, a pentylene group and a hexylene group.

The (b) diol(s) having at least one carbon chain comprising 3 or more carbon atoms may have a linear, branched or cyclic molecular structure, as long as it has the carbon chain comprising 3 or more carbon atoms.

The (b) diol(s) having at least one carbon chain comprising 3 or more carbon atoms may be a C₃₋₁₀ diol, preferably C₄₋₈ diol, and more preferably C₅₋₆ diol, comprising 2 hydroxy groups.

It is preferable that the (b) diol(s) having at least one carbon chain comprising 3 or more carbon atoms be selected from the group consisting of propylene glycol, dipropylene glycol, polypropylene glycol, butylene glycol, dibutylene glycol, polybutylene glycol, pentylene glycol, dipentylene glycol, hexylene glycol, dihexylene glycol, and a mixture thereof.

It is more preferable that the (b) diol(s) having at least one carbon chain comprising 3 or more carbon atoms be selected from the group consisting of propylene glycol, dipropylene glycol, butylene glycol, pentylene glycol, hexylene glycol, and a mixture thereof. Pentylene glycol is most preferable.

The amount of the (b) diol(s) having at least one carbon chain comprising 3 or more carbon atoms in the composition according to the present invention may be 20% by weight or more, preferably 25% by weight or more, and more preferably 30% by weight or more, relative to the total weight of the composition.

On the other hand, the amount of the (b) diol(s) having at least one carbon chain comprising 3 or more carbon atoms in the composition according to the present invention may be 60% by weight or less, preferably 55% by weight or less, and more preferably 50% by weight or less, relative to the total weight of the composition.

Thus, the amount of the (b) diol(s) having at least one carbon chain comprising 3 or more carbon atoms in the composition according to the present invention may be from 20% to 60% by weight, preferably from 25% to 55% by weight, and more preferably from 30% to 50% by weight, relative to the total weight of the composition.

### [Diol/Oil Weight Ratio]

The weight ratio of the amount of the (b) diol(s) having at least one carbon chain comprising 3 or more carbon atoms to the amount of the (a) oil(s) may be 4 or more, preferably 5 or more, and more preferably 6 or more.

On the other hand, the weight ratio of the amount of the (b) diol(s) having at least one carbon chain comprising 3 or more carbon atoms to the amount of the (a) oil(s) may be 20 or less, preferably 15 or less, and more preferably 10 or less.

Thus, the weight ratio of the amount of the (b) diol(s) having at least one carbon chain comprising 3 or more carbon atoms to the amount of the (a) oil(s) may be from 4 to 20, preferably from 5 to 15, and more preferably from 6 to 10.

### [Skin Care Active Agent]

In one embodiment, the composition according to the present invention comprises (c1) at least one skin care active ingredient. A single type of (c1) skin care active ingredient may be used, but two or more different types of (c1) skin care active ingredients may be used in combination.

It is preferable that the (c1) skin care active ingredient have a log P value of 4 or less, more preferably ranging from −4.5 to 4.0, and even more preferably from −4.0 to 3.5.

A log P value is a value for the base-ten logarithm of the apparent octan-1-ol/water partition coefficient. The log p values are known and are determined by a standard test which determines the concentration of the (c1) compound in octan-1-ol and water. The log P may be calculated according to the method described in the article by Meylan and Howard: Atom/Fragment contribution method for estimating octanol-water partition coefficients, J. Pharm. Sci., 84:83-92, 1995. This value may also be calculated using numerous commercially available software packages, which determine the log P as a function of the structure of a molecule. By way of example, mention may be made of the Epiwin software from the United States Environmental Agency.

The values may especially be calculated using the ACD (Advanced Chemistry Development) Solaris software V4.67; they may also be obtained from Exploring QSAR: hydrophobic, electronic and steric constants (ACS professional reference book, 1995). There is also an Internet site which provides estimated values (address: http://esc.syrres.com/interkow/kowdemo.htm).

The (c1) skin care active ingredient may be in the form of a salt. The salts of the (c1) skin care active ingredient include conventional non-toxic salts of said compounds, such as those formed from an acid or from a base.

It is preferable that the (c1) skin care active ingredient be a skin care cosmetic active ingredient, and more preferably a skin whitening ingredient or a skin anti-aging ingredient such as a skin anti-wrinkle ingredient.

As the (c1) skin care active ingredient, mention may be made of Vitamin B3 and derivatives.

Vitamin B3, also called vitamin PP, is a compound of the following formula (I):

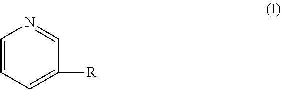

in which R may be —CONH₂ (niacinamide), —COOH (nicotinic acid or niacin), or CH₂OH (nicotinyl alcohol), —CO—NH—CH₂—COOH) (nicotinuric acid) or —CO—NH—OH (niconityl hydroxamic acid). Niacinamide is preferable.

Vitamin B3 derivatives that may be mentioned include, for example, nicotinic acid esters such as tocopherol nicotinate, amides derived from niacinamide by substitution of the hydrogen groups of —CONH₂, products from reaction with carboxylic acids and amino acids, esters of nicotinyl alcohol and of carboxylic acids such as acetic acid, salicyclic acid, glycolid acid or palmitic acid.

Mention may also be made of the following derivatives: 2-chloronicotinamide, 6-methylnicotinamide, 6-aminonicotinamide, N-methylnicotinamide, N,N-dimethylnicotinamide, N-(hydroxymethyl) nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine, methyl nicotinate and sodium nicotinate.

Other vitamin B3 derivatives that may also be mentioned include its inorganic salts, such as chlorides, bromides, iodides or carbonates, and its organic salts, such as the salts obtained by reaction with carboxylic acids, such as acetate, salicylate, glycolate, lactate, malate, citrate, mandelate, tartrate, etc.

As the (c1) skin care active ingredient, mention may be made of ascorbic acid and derivatives thereof.

Ascorbic acid is generally in L form, since it is usually extracted from natural products.

On account of its chemical structure (a-keto lactone) which makes it very sensitive to certain environmental parameters such as light, heat and aqueous media, it may be advantageous to use the ascorbic acid in the form of a derivative or an analog chosen, for example, from saccharide esters of ascorbic acid or metal salts of phosphoryl ascorbic acid, alkali metal salts, esters and sugars.

The saccharide esters of ascorbic acid that may be used in the present invention are especially the glycosyl, mannosyl, fructosyl, fucosyl, galactosyl, N-acetylglucosamine and N-acetylmuramic derivatives of ascorbic acid, and a mixture thereof, and more especially ascorbyl glucoside such as ascorbyl-2 glucoside, 2-O-α-D-glucopyranosyl L-ascorbic acid or 6-O-β-D-galactopyranosyl L-ascorbic acid. The latter compounds and processes for preparing them are described in particular in documents EP-A-0 487 404, EP-A-0 425 066 and JP 05 213 736.

As regards to the metal salt of phosphoryl ascorbic acid, it may be chosen from alkali metal, and especially sodium, ascorbyl phosphates, alkaline-earth metal ascorbyl phosphates and transition metal ascorbyl phosphates.

It is also possible to use ascorbic acid precursors such as active agent amides and active agent saccharide derivatives, which respectively involve proteases or peptidases and glycosidases as enzymes for releasing ascorbic acid in situ. Such compounds are described in patent EP 0 667 145.

The active agent saccharide derivatives are especially chosen from C₃ to C₆ saccharide derivatives. They are especially chosen from glucosyl, mannosyl, fructosyl, fucosyl, N-acetylglucosamine, galactosyl and N-acetylgalactosamine derivatives, N-acetylmuramic acid derivatives and sialic acid derivatives, and a mixture thereof.

The second ascorbic acid precursors may be chosen from derivatives that are hydrolyzed by other enzymes, for example by esterases, phosphatases, sulfatases, etc. According to the present invention, the second active agent precursors may be chosen, for example, from phosphates; sulfates; palmitates; acetates; propionates; ferulates, and, in general, active agent alkyl or acyl esters; acyl or alkyl ethers. The acyl and alkyl radicals in particular contain from 1 to 30 carbon atoms. As the skin care active ingredient, mention may be made of 3-O-ethyl ascorbic acid.

In particular, the second precursor may be an ester derived from the reaction with a mineral acid such as a sulfate or a phosphate to react with a sulfatase or phosphatase on contact with the skin, and the second precursor may be an acyl or alkyl ester derived from the reaction with an organic acid, for instance palmitic acid, acetic acid, propionic acid, nicotinic acid, 1,2,3-propanetricarboxylic acid or ferulic acid to react with a specific skin esterase.

Other derivatives are described, for example, in patent EP 1 430 883.

The ascorbic acid analogs are, more particularly, its salts, especially alkali metal salts, for example sodium ascorbate, its esters, especially such as its acetic, propionic or palmitic esters, or its sugars, especially such as glycosyl ascorbic acid.

As the (c1) skin care active ingredient, mention may be made of resorcinol derivatives.

The resorcinol derivative may preferably be 4-position substituted derivatives, such as 4-alkylresorcinols, more preferably phenyl ethyl resorcinol, 4-n-butylresorcinol and 4-(tetrahydro-2H-pyran-4-yl)benzene-1,3-diol, and in particular phenyl ethyl resorcinol because of its whitening effect. Phenyl ethyl resorcinol is also referred to as 4-(1-phenylethyl)-1,3-benzenediol and represented by the following chemical formula. Phenyl ethyl resorcinol can be obtained, for example, from Symrise Corp (the product name: Symwhite 377®).

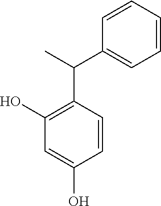 [SMILES: CC(c1ccccc1)c1ccc(O)cc1O]

As other examples of resorcinol derivatives, mention may be made of: 2-methylresorcinol, 5-methylresorcinol, 4-methylresorcinol, 4-ethylresorcinol, 2,5-dimethylresorcinol, 4,5-dimethylresorcinol, 2,4-dimethyl-1,3-benzenediol, 3,5-dihydroxybenzylamine, 5-methoxyresorcinol, 3,5-dihydroxybenzyl alcohol, 2-methoxyresorcinol, 4-methoxyresorcinol, 3,5-dihydroxytoluene monohydrate, 4-chlororesorcinol, 2-chlororesorcinol, 2′,4′-dihydroxyacetophenone, 3′,5′-dihydroxyacetophenone, 2,6-dihydroxy-4-methylbenzaldehyde, 4-propylresorcinol, 2,4-dihydroxy-1,3,5-trimethylbenzene, 3,5-dihydroxybenzamide, 2,6-dihydroxybenzamide, 2,4-dihydroxybenzamide, 2,4-dihydroxybenzoic acid, 2,6-dihydroxybenzoic acid, 3,5-dihydroxybenzoic acid, 2,6-dihydroxy-4-methylbenzyl alcohol, 3,5-dihydroxyanisole hydrate, 4-aminoresorcinol hydrochloride, 2-aminoresorcinol hydrochloride, 5-aminobenzene-1,3-diol hydrochloride, 2′,4′-dihydroxypropiophenone, 2′,4′-dihydroxy-3′-methylacetophenone, (2,4-dihydroxyphenyl) acetone, (3,5-dihydroxyphenyl) acetone, 2,6 -dihydroxy-4′-methylacetophenone, 4-n-butylresorcinol, 2,4-diethyl-1,3-benzenediol, 3,5-dihydroxy-4-methylbenzoic acid, 2,6-dihydroxy-4-methylbenzoic acid, 2,4-dihydroxy-6-methylbenzoic acid, 3,5-dihydroxyphenylacetic acid, 2-ethyl-5-methoxybenzene-1,3-diol, 4-amino-3,5-dihydroxybenzoic acid, 3,5-dihydroxyacetophenone monohydrate, 3,5-dihydroxybenzylamine hydrochloride, 4,6-dichlororesorcinol, 2′,4′-dihydroxy-3′-methylpropiophenone, 1-(3-ethyl-2,6-dihydroxyphenyl) ethan-1-one, 2′,6′-dihydroxy-4′-methoxyacetophenone, 1-(2,6-dihydroxy-3-methoxyphenyl) ethan-1-one, 3 (2,4-dihydroxyphenylpropionic acid, and 2,4-dihydroxy-3,6-dimethylbenzoic acid.

As the (c1) skin care active ingredient, mention may be made of C-glycoside derivatives.

The C-glycoside derivative(s) that may be present in the composition in accordance with the present invention are chosen from the compounds of general formula (II) below:

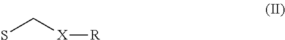

in which:
R denotes an unsubstituted linear C₁-C₄ and especially C₁-C₂ alkyl radical, in particular methyl;
S represents a monosaccharide chosen from D-glucose, D-xylose, N-acetyl-D-glucosamine and L-fucose, and in particular D-xylose;
X represents a group chosen from —CO—, —CH(OH)— and —CH(NH₂)— and preferentially a —CH(OH)— group;
and also the cosmetically acceptable salts thereof, solvates thereof such as hydrates, and optical isomers thereof.

As nonlimiting illustrations of C-glycoside derivatives that are more particularly suitable for use in the present invention, mention may be made especially of the following derivatives:
C-beta-D-xylopyranoside-n-propan-2-one;
C-alpha-D-xylopyranoside-n-propan-2-one;
C-beta-D-xylopyranoside-2-hydroxypropane;
C-alpha-D-xylopyranoside-2-hydroxypropane;
1-(C-beta-D-glucopyranosyl)-2-hydroxypropane;
1-(C-alpha-D-glucopyranosyl)-2-hydroxpropane;
1-(C-beta-D-glucopyranosyl)-2-aminopropane;
1-(C-alpha-D-glucopyranosyl)-2-aminopropane;
3′-(acetamido-C-beta-D-glucopyranosyl)propan-2′-one;
3′-(acetamido-C-alpha-D-glucopyranosyl)propan-2′-one;
1-(acetamido-C-beta-D-glucopyranosyl)-2-hydroxpropane;
1-(acetamido-C-beta-D-glucopyranosyl)-2-aminopropane;
   and also the cosmetically acceptable salts thereof, solvates thereof such as hydrates, and optical isomers thereof.

According to a particular embodiment, C-beta-D-xylopyranoside-2-hydroxypropane or C-alpha-D-xylopyranoside-2-hydroxypropane, and better still C-beta-D-xylopyranoside-2-hydroxypropane, may be advantageously used for the preparation of the composition according to the present invention.

According to a particular embodiment, a C-glycoside derivative that is suitable for use in the present invention may advantageously be hydroxypropyltetrahydropyrantriol, also known as C-beta-D-xylopyranoside-2-hydroxpropane, sold especially as a solution at 30% by weight in a water/propylene glycol mixture (60/40) under the name Mexoryl SBB® by Chimex. According to one embodiment, the C-glycoside derivative is in the form of a solution in which it is present in an amount of 30% by weight relative to the total weight of the solution, the remainder being a mixture of water and propylene glycol.

The salts of the C-glycoside derivatives that are suitable for use in the present invention may comprise conventional physiologically acceptable salts of these compounds, such as those formed from organic or mineral acids. Examples that may be mentioned include the salts of mineral acids, such as sulfuric acid, hydrochloric acid, hydrobromic acid, hydroiodic acid, phosphoric acid and boric acid. Mention may also be made of the salts of organic acids, which may comprise one or more carboxylic, sulfonic or phosphonic acid groups. They may be linear, branched or cyclic aliphatic acids, or alternatively aromatic acids. These acids may also comprise one or more heteroatoms chosen from 0 and N, for example in the form of hydroxyl groups. Mention may be made especially of propionic acid, acetic acid, terephthalic acid, citric acid and tartaric acid.

The solvates that are acceptable for the compounds described above comprise conventional solvates such as those formed during the final step of preparation of said compounds due to the presence of solvents. Examples that may be mentioned include solvates due to the presence of water or of linear or branched alcohols, such as ethanol or isopropanol.

A C-glycoside derivative that is suitable for use in the present invention may especially be obtained via the synthetic method described in document WO 02/051 828, the content of which is incorporated herein by reference.

As the (c1) skin care active ingredient, mention may be made of salicylic acid and derivatives thereof. The derivatives of salicylic acid may be represented by the formula (III):

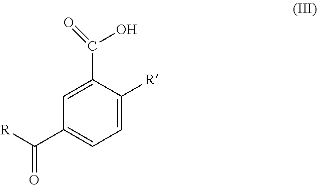

wherein
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from (a) halogen atoms, (b) the trifluoromethyl group, (c) hydroxyl groups in free form or esterified with an acid containing from 1 to 6 carbon atoms, or (d) a carboxyl function in free form or esterified with a lower alcohol containing from 1 to 6 carbon atoms; and
R′ is a hydroxyl group. The salicylic acid derivative may be in the form of a salt derived from an inorganic or organic base.

As the (c1) skin care active ingredient, mention may be made of tranexamic acid and derivatives thereof such as esters and amides of tranexamic acid.

As examples of derivatives of tranexamic acid, mention may be made of dimers of tranexamic acid (such as hydrochloric acid trans-4-(trans-aminomethylcyclohexanecarbonyl) aminomethylcyclohexane carboxylic acid), esters of tranexamic acid and hydroquinone (such as 4′-hydroxyphenyl trans-4-aminomethylcyclohexane carboxylate), esters of tranexamic acid and gentisic acid (such as 2-(trans-4-aminomethylcyclohexanecarbonyloxy)-5-hydroxybenzoic acid and salts thereof), tranexamic amides (such as trans-4-aminomethylcyclohexanecarboxylic acid methylamide and salts thereof, trans-4-(p-methoxybenzoyl)aminomethylcyclohexane carboxylic acid and salts thereof, and trans-4-guanidinomethylcyclohexane carboxylic acid and salts thereof), and the like.

It is even more preferable that the (c1) skin care active ingredient be selected from the group consisting of ascorbyl glucoside, 3-O-ethyl ascorbic acid, tranexamic acid, hydroxypropyl tetrahydropyrantriol, niacinamide, salicylic acid, phenylethyl resorcinol, and a mixture thereof.

The amount of the (c1) skin care active ingredient in the composition according to the present invention may be 0.01% by weight or more, preferably 0.05% by weight or more, and more preferably 0.1% by weight or more, relative to the total weight of the composition.

On the other hand, the amount of the (c1) skin care active ingredient in the composition according to the present invention may be 25% by weight or less, preferably 20% by weight or less, and more preferably 15% by weight or less, relative to the total weight of the composition.

Thus, the amount of (c1) skin care active ingredient in the composition according to the present invention may range from 0.01% to 25% by weight, preferably from 0.05% to 20% by weight, more preferably from 0.1% to 15% by weight, relative to the total weight of the composition.

### [Cellulose Compound]

In another embodiment, the composition according to the present invention comprises (c2) at least one cellulose compound. Two or more (c2) cellulose compounds may be used.

The (c2) cellulose compounds may be anionic, cationic, amphoteric or non-ionic polymers.

The term “cellulose” compounds mean, according to the present invention, any polysaccharide compound having in its structure sequences of glucose residues linked together via β-1,4 linkages, including unsubstituted celluloses, as well as anionic, cationic, amphoteric and non-ionic cellulose derivatives.

Thus, the (c2) cellulose compounds may be chosen from, for example, unsubstituted celluloses, including in a microcrystalline form, as well as cellulose ethers, cellulose esters, cellulose ester ethers and mixtures thereof. Among the cellulose esters, mention may be made of inorganic esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/inorganic esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

According to one particular embodiment of the present invention, the (c2) cellulose compound is non-associative. The “non-associative” cellulose compounds are cellulose polymers which do not comprise any fatty chain, and preferably do not comprise any C₁₀-C₃₀ chain, in their structure.

According to a first variant, the (c2) cellulose compound is non-ionic. Mention may be made of (C₁-C₄)alkylcelluloses such as methylcelluloses and ethylcelluloses for example Ethocel standard 100 Premium from Dow Chemical; (poly)hydroxy(C₁-C₄)alkylcelluloses such as hydroxymethylcelluloses, hydroxyethylcelluloses for example Natrosol 250 HHR provided by Aqualon and hydroxypropylcelluloses for example Klucel EF from Aqualon; mixed (poly)hydroxy(C₁-C₄)alkyl-(C₁-C₄)alkylcelluloses such as hydroxypropylmethylcelluloses for example Methocel E4M from Dow Chemical; hydroxyethylmethylcelluloses, hydroxyethylethylcelluloses for example Bermocoll E 481 FQ from Akzo Noble and hydroxybutylmethylcelluloses.

According to a second variant, the (c2) cellulose compound is anionic. Among the anionic cellulose ethers, mention may be made of (poly) carboxy (C₁-C₄)alkylcelluloses and salts thereof. Examples that may be mentioned include carboxymethylcelluloses, carboxymethylmethylcelluloses for example Blanose 7M from the company Aqualon and carboxymethylhydroxyethylcelluloses, and the sodium salts thereof.

According to a third variant, the (c2) cellulose compound is cationic. Among the cationic cellulose, mention may be made of cationic cellulose derivatives such as cellulose copolymers or cellulose derivatives grafted with a water-soluble quaternary ammonium monomer, and described in particular in patent U.S. Pat. No. 4,131,576, such as (poly)hydroxy(C₁-C₄)alkyl celluloses, for instance hydroxymethyl-, hydroxyethyl- or hydroxypropylcelluloses grafted in particular with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by the company National Starch.

According to another particular embodiment of the present invention, the (c2) cellulose compound is associative. The “associative” cellulose compounds are cellulose polymers which comprises any fatty chain, and preferably comprises a C₁₀-C₃₀ chain, in their structure.

The associative cellulose compounds may be cationic. Among these cationic associative cellulose compounds preferred are quaternized (poly) hydroxyethylcelluloses modified by groups comprising at least one fatty chain, such as alkyl, arylalkyl or alkylaryl groups comprising at least 8 carbon atoms, or mixtures of these. The alkyl radicals carried by the quaternized celluloses orhydroxyethylcelluloses above preferably comprise from 8 to 30 carbon atoms. The arylradicals preferably denote phenyl, benzyl, naphthyl or anthryl groups. There may be indicated, as examples of quaternized alkylhydroxyethylcelluloses having C₈-C₃₀ fatty chains, the products Quatrisoft LM 200a, Quatrisoft LM-X 529-18-Aa, Quatrisoft LM-X 529-18-Ba (C₁₂ alkyl) and Quatrisoft LM-X 529-8a (C₁₈ alkyl), sold by Aqualon, the products Crodacel QMa, Crodacel QLa (C₁₂ alkyl) and Crodacel QSa (C₁₈ alkyl), sold by Croda and the product Softcat SL 100a sold by Aqualon.

The associative cellulose compounds may be non-ionic. Among these non-ionic associative cellulose compounds preferred are celluloses or their derivatives, modified by groups comprising at least one fattychain, such as alkyl, arylalkyl or alkylaryl groups or their mixtures, where the alkyl groups are C₈-C₃₀ alkyl groups and in particular: nonionic alkylhydroxyethylcelluloses, such as the products Natrosol Plus Grade 330 CS and Polysurf 67 (C₁₆ alkyl) sold by Aqualon; nonionic nonoxynyl hydroxyethylcelluloses, such as the product Amercell HM-1500 sold by Amerchol; nonionic alkylcelluloses, such as the product Bermocoll EHM 100 sold by Berol Nobel.

The (c2) cellulose compounds may preferably be non-ionic or anionic, and free of fatty chain (non-associative, non-ionic or anionic alkylcellulose polymer). Preferably, the (c2) cellulose compounds which may be useful in the present invention are chosen from non-ionic cellulose ethers such as (C₁-C₄)alkylcelluloses, (poly)hydroxy(C₁-C₄)alkylcelluloses (poly)hydroxy(C₁-C₄)alkyl-(C₁-C₄)alkylcelluloses, such as hydroxypropylmethylcelluloses hydroxyethylmethylcelluloses hydroxyethylethylcelluloses, hydroxybutylmethylcelluloses hydroxymethylcelluloses, hydroxyethylcelluloses and hydroxypropylcelluloses; and anionic cellulose ethers such as carboxymethylcelluloses, in particular sodium carboxymethylcellulose.

It is preferable that the (c2) cellulose compound be selected from the group consisting of unmodified cellulose and nonionic or anionic cellulose ethers.

It is more preferable that the (c2) cellulose compound be selected from the group consisting of cellulose, cellulose gum, hydroxymethylcellulose, hydroxyethylcellulose, hydroxypropylmethylcellulose, sodium carboxymethylcellulose, and a mixture thereof.

The amount of the (c2) cellulose compound(s) in the composition according to the present invention may be 0.01% by weight or more, preferably 0.05% by weight or more, and more preferably 0.1% by weight or more, relative to the total weight of the composition.

On the other hand, the amount of the (c2) cellulose compound(s) in the composition according to the present invention may be 40% by weight or less, preferably 35% by weight or less, and more preferably 30% by weight or less, relative to the total weight of the composition.

Thus, the amount of the (c2) cellulose compound(s) in the composition according to the present invention may be from 0.01% to 40% by weight, preferably from 0.05% to 35% by weight, and more preferably from 0.01% to 30% by weight, relative to the total weight of the composition.

### [Water]

The composition according to the present invention comprises (d) water.

If the composition according to the present invention comprises the (c1) skin care active ingredient, the amount of the (d) water is as follows.

The amount of the (d) water in the composition according to the present invention may be 30% by weight or more, preferably 35% by weight or more, and more preferably 40% by weight or more, relative to the total weight of the composition.

The amount of the (d) water in the composition according to the present invention may be 60% by weight or less, preferably 55% by weight or less, and more preferably 50% by weight or less, relative to the total weight of the composition.

The amount of (d) water in the composition according to the present invention may range from 30% to 60% by weight, preferably from 35% to 55% by weight, more preferably from 40% to 50% by weight, relative to the total weight of the composition.

On the other hand, if the composition according to the present invention comprises the (c2) cellulose compound, the amount of the (d) water is as follows.

The amount of the (d) water in the composition according to the present invention may be 10% by weight or more, preferably 15% by weight or more, and more preferably 20% by weight or more, relative to the total weight of the composition.

The amount of the (d) water in the composition according to the present invention may be 80% by weight or less, preferably 75% by weight or less, and more preferably 70% by weight or less, relative to the total weight of the composition.

The amount of (d) water in the composition according to the present invention may range from 10% to 80% by weight, preferably from 15% to 75% by weight, more preferably from 20% to 60% by weight, relative to the total weight of the composition.

### [Optional Ingredients]

The composition according to the present invention may further comprise at least one optional ingredient usually used in the field of cosmetics, chosen, for example, film-forming polymers, solvents, dyes, pigments, UV filters, antioxidants, preserving agents, pH adjusting agents, and mixtures thereof.

The composition according to the present invention may include one or several cosmetically acceptable organic solvents, which may be alcohols: in particular monovalent alcohols such as ethyl alcohol, isopropyl alcohol, benzyl alcohol, and phenylethyl alcohol; diols other than the ingredient (b), such as ethylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ether, and butylene glycol monomethyl, monoethyl, and monobutyl ethers.

The amount of the organic solvent(s) in the composition according to the present invention may be 0.01% by weight or more, preferably 0.1% by weight or more, and more preferably 1% by weight or more, relative to the total weight of the composition.

On the other hand, the amount of the organic solvent(s) in the composition according to the present invention may be 30% by weight or less, preferably 20% by weight or less, and more preferably 10% by weight or less, relative to the total weight of the composition.

Thus, the amount of the organic solvent(s) in the composition according to the present invention may range from 0.01% to 30% by weight, preferably from 0.1% to 20% by weight, and more preferably from 1% to 10% by weight, relative to the total weight of the composition.

### (Preparation)

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

### [Form]

The form of the composition according to the present invention is not limited.

The composition according to the present invention can be thermodynamically stable, and may be considered as a single phase composition.

The composition according to the present invention can be transparent.

The transparency may be measured by measuring the turbidity (for example, turbidity can be measured with a 2100Q (marketed by Hach Company) having a round cell (25 mm in diameter and 60 mm height) and a tungsten filament lamp which can emit visible light (between 400 and 800 nm, preferably from 400 to 500 nm). The measurement can be performed on the undiluted composition. The blank may be determined with distilled water.

The composition according to the present invention has a turbidity of 120 NTU or less, preferably 90 NTU or less and more preferably 60 NTU or less.

### [Process and Use]

It is preferable that the composition according to the present invention be a cosmetic or dermatological composition, preferably a skin cosmetic composition, and more preferably a skin care cosmetic composition.

The composition according to the present invention can be used for a non-therapeutic process, such as a cosmetic process, for treating a skin, by being applied to the skin.

Thus, the present invention also relates to a cosmetic process for treating a keratin substance such as skin, comprising the step of applying the composition according to the present invention to the keratin substance.

The present invention may also relate to a use of the composition according to the present invention as a cosmetic product or in a cosmetic product such as skin care products.

In other words, the composition according to the present invention can be used, as it is, as a cosmetic product. Alternatively, the composition according to the present invention can be used as an element of a cosmetic product. For example the composition according to the present invention can be added to or combined with any other elements to form a cosmetic product.

The skin care product may be a lotion, a cream, a serum, and the like.

Another aspect of the present invention also relates to a process for preparing a composition, comprising a step of mixing:
(a) at least one oil;
(b) at least one diol having at least one carbon chain comprising 3 or more carbon atoms;
(c1) at least one skin care active ingredient; and
(d) water.

It is preferable that the mixing step be performed by a so-called low energy process without a special mechanical stirrer, such as a homogenizer which uses a large amount of energy. The low energy process can be performed by simply gently stirring the ingredients (a) to (d).

Another aspect of the present invention relates to a use of:
(b) at least one diol having at least one carbon chain comprising 3 or more carbon atoms in a composition, comprising
(a) at least one oil,
(c1) at least one skin care active ingredient, and
(d) water
in order to enhance or improve the penetration of the (c1) skin care active ingredient.

The present invention also relates to a use of:
(b) at least one diol having at least one carbon chain comprising 3 or more carbon atoms as a skin penetration enhancer for
(c1) at least one skin care active ingredient,
in a composition comprising,
(a) at least one oil, and
(d) water.

Another aspect of the present invention also relates to a process for preparing a composition, comprising a step of mixing:
(a) at least one oil;
(b) at least one diol having at least one carbon chain comprising 3 or more carbon atoms;
(c2) at least one cellulose compound; and
(d) water.

It is preferable that the mixing step be performed by a so-called low energy process without a special mechanical stirrer, such as a homogenizer which uses a large amount of energy. The low energy process can be performed by simply gently stirring the ingredients (a) to (d).

Another aspect of the present invention relates to a use of
(c2) at least one cellulose compound
in a composition comprising:
(a) at least one oil;
(b) at least one diol having at least one carbon chain comprising 3 or more carbon atoms; and
(d) water
in order to enhance glow of the skin on which the composition according to the present invention has been applied.

The present invention also relates to a use of
(c2) at least one cellulose compound
as a skin glow enhancer for a composition comprising:
(a) at least one oil;
(b) at least one diol having at least one carbon chain comprising 3 or more carbon atoms; and
(d) water.

It is preferable that the above composition in the use and process according to the present invention be in the form of a micro-emulsion, and more preferably an O/W micro-emulsion.

The above explanations regarding the ingredients (a) to (d), as well as the optional ingredients, for the composition according to the present invention can apply to those for the uses and processes according to the present invention. The explanations regarding the preparation and forms of the composition according to the present invention can also apply to those of the composition recited in the above uses and processes.

### EXAMPLES

The present invention will be described in more detail by way of examples which, however, should not be construed as limiting the scope of the present invention.

### Examples 1A-6A and Comparative Examples 1A-12A

The following compositions according to Examples 1A-6A and Comparative Examples 1A-12A shown in Tables 1A-3A, were prepared by mixing the ingredients shown in Tables 1A-3A as follows. The symbols “(a)”, “(b)”, “(c1)”, and “(d)” in Tables 1A-3A correspond to those in the claims. The numerical values for the amounts of the ingredients shown in Tables 1A-3A are all based on “% by weight” as active raw materials.

TABLE 1A

| | Ingredients | Log P | Ex. 1A | Ex. 2A | Ex. 3A | Ex. 4A | Ex. 5A | Ex. 6A |
|---|---|---|---|---|---|---|---|---|
| (d) | Water | | qsp 100 | qsp 100 | qsp 100 | qsp 100 | qsp 100 | qsp 100 |
| | Hydroxyethylcellulose | | 0.2 | 0.2 | 0.2 | 0.2 | 0.2 | 0.2 |
| (b) | Pentylene Glycol | | 40 | 40 | 40 | 40 | 40 | 40 |
| (a) | Isopropyl lauroyl Sarcosinate | | 10 | 10 | 10 | 10 | 10 | 10 |
| (c1) | Ascorbyl Glucoside | −3.4 | 1 | — | — | — | — | — |
| | Tranexamic Acid | −1.6 | — | 1 | — | — | — | — |
| | Hydroxypropyl Tetrahydropyrantriol | −1.5 | — | = | 10 | — | — | — |
| | Niacinamide | −0.37 | — | — | — | 5 | — | — |
| | Salicylic Acid | 2.2 | — | — | — | — | 0.2 | — |
| | Phenylethyl Resorcinol | 3.07 | — | — | — | — | — | 0.3 |

| | | | | | | |
|---|---|---|---|---|---|---|
| Penetration Amount of | 68.2 | 79.0 | 50.1 | 75.3 | 81.9 | 1.5 |
| Active Ingredient (%) | | | | | | |

TABLE 2A

| | | | Comp. | Comp. | Comp. | Comp. | Comp. | Comp. |
|---|---|---|---|---|---|---|---|---|
| | Ingredients | Log P | Ex. 1A | Ex. 2A | Ex. 3A | Ex. 4A | Ex. 5A | Ex. 6A |
| (d) | Water | | qsp 100 | qsp 100 | qsp 100 | qsp 100 | qsp 100 | qsp 100 |
| | Hydroxyethylcellulose | | 0.2 | 0.2 | 0.2 | 0.2 | 0.2 | 0.2 |
| | Acrylates/C10-30 Alkyl Acrylate | | 0.5 | 0.5 | 0.5 | 0.5 | 0.5 | 0.5 |
| | Crosspolymer | | | | | | | |
| (a) | Isopropyl lauroyl Sarcosinate | | 10 | 10 | 10 | 10 | 10 | 10 |
| (c1) | Ascorbyl Glucoside | −3.4 | 1 | — | — | — | — | — |
| | Tranexamic Acid | −1.6 | — | 1 | — | — | — | — |
| | Hydroxypropyl Tetrahydropyrantriol | −1.5 | — | — | 10 | — | — | — |
| | Niacinamide | −0.37 | — | — | — | 5 | — | — |
| | Salicylic Acid | 2.2 | — | — | — | — | 0.2 | — |
| | Phenylethyl Resorcinol | 3.07 | — | — | — | — | — | 0.3 |

| | | | | | | |
|---|---|---|---|---|---|---|
| Penetration Amount of | 0 | 0 | 0.2 | 19.7 | 77.9 | 0 |
| Active Ingredient (%) | | | | | | |

TABLE 3A

| | | | Comp. | Comp. | Comp. | Comp. | Comp. | Comp. |
|---|---|---|---|---|---|---|---|---|
| | Ingredients | Log P | Ex. 7A | Ex. 8A | Ex. 9A | Ex. 10A | Ex. 11A | Ex. 12A |
| (d) | Water | | qsp 100 | qsp 100 | qsp 100 | qsp 100 | qsp 100 | qsp 100 |
| | Hydroxyethylcellulose | | 0.2 | 0.2 | 0.2 | 0.2 | 0.2 | 0.2 |
| | Ethylcellulose | | 0.5 | 0.5 | 0.5 | 0.5 | 0.5 | 0.5 |
| (a) | Isopropyl lauroyl Sarcosinate | | 10 | 10 | 10 | 10 | 10 | 10 |
| (c1) | Ascorbyl Glucoside | −3.4 | 1 | — | — | — | — | — |
| | Tranexamic Acid | −1.6 | — | 1 | — | — | — | — |
| | Hydroxypropyl Tetrahydropyrantriol | −1.5 | — | — | 10 | — | — | — |
| | Niacinamide | −0.37 | — | — | — | 5 | — | — |
| | Salicylic Acid | 2.2 | — | — | — | — | 0.2 | — |
| | Phenylethyl Resorcinol | 3.07 | — | — | — | — | — | 0.3 |

| | | | | | | |
|---|---|---|---|---|---|---|
| Penetration Amount of | 0 | 0 | 0.6 | 7.5 | 42.2 | 0 |
| Active Ingredient (%) | | | | | | |

### [Evaluation]

### (Penetration Amount of Active Ingredient)

The penetration test experiments were performed in a Franz diffusion cell (PermeGear) with a Strat-M membrane (Merck, surface area: 1.77 cm²). This equipment was composed of a donor part and a receptor part with the Strat-M membrane between the donor and receptor parts. The receptor part with a predetermined volume was filled with a receiving solution (0.25 wt % Tween 80/deionized water) maintained at a temperature of 32° C., which was continuously stirred with a small magnetic bar. Each of the compositions according to Examples 1A-6A and Comparative Examples 1A-12A was spread with a spatula on the membrane of the donor part with an amount of 20 mg/cm². After 1 hour, 200 μl of the receiving solution was withdrawn from the receptor part, while providing the same amount of a new receiving solution to the receptor part to maintain the same penetration conditions. The withdrawn receiving solution was analyzed by HPLC to determine the amount of the active ingredient in the receiving solution. The percentage of the active ingredient that passed through the membrane was calculated by dividing the detected amount of the active ingredient in the receiving solution by the amount of the active ingredient spread on the membrane.

The results are shown in the line of “Penetration Amount of Active Ingredient” in Tables 1A-3A.

### [Summary]

Tables 1A-3A demonstrate that a composition comprising (a) at least one oil, (b) at least one diol having at least one carbon chain comprising 3 or more carbon atoms, (c1) at least one skin care active ingredient, and (d) water can provide enhanced skin penetration of the (c1) skin care active ingredient.

Tables 1A-3A also demonstrate that the use of (b) at least one diol having at least one carbon chain comprising 3 or more carbon atoms in a composition comprising (a) at least one oil, (c1) at least one skin care active ingredient, and (d) water can enhance or improve the penetration of the (c1) skin care active ingredient.

### Examples 1B-5B and Comparative Examples 1B-8B

The following compositions according to Examples 1B-5B and Comparative Examples 1B-8B shown in Table 1B, were prepared by mixing the ingredients shown in Table 1B as follows. The numerical values for the amounts of the ingredients shown in Table 1B are all based on “% by weight” as active raw materials.

TABLE 1B

| | | | | | | Comp. | Comp. | Comp. | Comp. | Comp. | Comp. | Comp. | Comp. |
|---|---|---|---|---|---|---|---|---|---|---|---|---|---|
| Ingredients | Ex. 1B | Ex. 2B | Ex. 3B | Ex. 4B | Ex. 5B | Ex. 1B | Ex. 2B | Ex. 3B | Ex. 4B | Ex. 5B | Ex. 6B | Ex. 7B | Ex. 8B |
| Isopropyl Lauroyl | 10 | 10 | 10 | 10 | 10 | 10 | 10 | 10 | 10 | 10 | — | 10 | — |
| Sarcosinate | | | | | | | | | | | | | |
| Pentylene Glycol | 35 | 35 | 35 | 35 | 35 | 35 | 35 | 35 | 35 | 35 | 35 | — | — |
| Water | qsp 100 | qsp 100 | qsp 100 | qsp 100 | qsp 100 | qsp 100 | qsp 100 | qsp 100 | qsp 100 | qsp 100 | qsp 100 | qsp 100 | qsp 100 |
| Hydroxyethyl | 0.5 | — | — | — | — | — | — | — | — | — | 0.5 | 0.5 | 0.5 |
| cellulose | | | | | | | | | | | | | |
| Hydroxypropyl | — | 0.5 | — | — | — | — | — | — | — | — | — | — | — |
| cellulose | | | | | | | | | | | | | |
| Hydroxypropylmethyl | — | — | 0.5 | — | — | — | — | — | — | — | — | — | — |
| cellulose | | | | | | | | | | | | | |
| Cellulose Gum | — | — | — | 0.5 | — | — | — | — | — | — | — | — | — |
| Cellulose | — | — | — | — | 25.0 | — | — | — | — | — | — | — | — |
| Sclerotium Gum | — | — | — | — | — | 0.5 | — | — | — | — | — | — | — |
| Hydroxypropyl Guar | — | — | — | — | — | — | 0.5 | — | — | — | — | — | — |
| Carbomer | — | — | — | — | — | — | — | 0.5 | — | — | — | — | — |
| Acrylates Copolymer | — | — | — | — | — | — | — | — | 1.0 | — | — | — | — |
| Potassium Hydroxide | — | — | — | — | — | — | — | 0.48 | — | — | — | — | — |
| Aspect | Good | Good | Good | Good | Good | Good | Good | Good | Good | Good | Good | Poor | Good |
| Transparency | Good | Good | Good | Good | Good | Fair | Good | Good | Good | Good | Good | NA | Good |
| Freshness | 3.50 | 3.50 | 3.50 | 4.50 | 4.00 | 3.00 | 3.50 | 3.50 | 3.50 | 3.50 | 4.00 | 3.50 | 4.00 |
| Glow | 4.50 | 3.00 | 3.00 | 3.00 | 4.00 | 1.00 | 1.00 | 1.00 | 1.00 | 1.00 | 1.00 | 1.00 | 1.00 |

NA: Not Available

### [Evaluations]

### (Aspect)

The aspect of each of the compositions according to Examples 1B-6B and Comparative Examples 1B-8B was evaluated by visual observation of the composition which was kept at 55° C. for 2 weeks and at 4° C. for 2 weeks, based on the following criteria.
Good: Uniform
Poor: Non-Uniform (phase separation was recognized)

The results are shown in Table 1B.

### (Transparency)

The transparency of each of the compositions according to Examples 1B-6B and Comparative Examples 1B-8B was evaluated by visual observation of the composition which was kept at 55° C. for 2 weeks and at 4° C. for 2 weeks, based on the following criteria.
Good: Transparent
Fair: Translucent
Poor: Opaque

The results are shown in Table 1B.

### (Freshness)

Each of the compositions according to Examples 1B-6B and Comparative Examples 1B-8B, in an amount of 100 μm, was applied onto the forearm of panelists, and massaged with the fingers lightly. The freshness during those steps was evaluated based on the following criteria.
Very Fresh: 5
Fresh: 4
Slightly Fresh: 3
Little Fresh: 2
Not Fresh: 1

The average scores tested by 5 skincare evaluation experts are shown in Table 1B.

### (Glow)

Each of the compositions according to Examples 1B-6B and Comparative Examples 1B-8B, in an amount of 45 μm, was evenly applied onto the area with 3.0 cm*3.0 cm (5 μm/cm²) on the forearm of panelists, and left for 1.5 hours. The glow of the above area was evaluated based on the following criteria.
Strongly Glowing: 5
Glowing: 4
Slightly Glowing: 3
Faintly Glowing: 2
Not Glowing: 1

The average scores tested by 5 skincare evaluation experts are shown in Table 1B.

### [Summary]

The compositions according to Examples 1B-6B were able to provide enhanced glow of the skin, while providing fresh feeling to touch, when being used.

The compositions according to Examples 1B-6B were uniform and transparent.

On the other hand, the composition according to Comparative Examples 1B-8B could not provide enhanced glow of the skin. However, they were able to provide fresh feeling to touch, when being used.

In addition, the composition according to Comparative Example 7B was not uniform.

Table 1B demonstrates that a combination of (a) at least one oil, (b) at least one diol having at least one carbon chain comprising 3 or more carbon atoms, (c2) at least one cellulose compound, and (d) water is necessary to provide a composition which can enhance glow of the skin, as compared to a case in which the ingredient (c2) is not used, while providing fresh texture when being used.

Table 1B also demonstrates that the use of (c2) at least one cellulose compound in a composition comprising (a) at least one oil, (b) at least one diol having at least one carbon chain comprising 3 or more carbon atoms, and (d) water can enhance glow of the skin on which the composition according to the present invention has been applied.

1. A composition, comprising:
(a) at least one oil;
(b) at least one diol having at least one carbon chain comprising 3 or more carbon atoms;
(c) (c1) at least one skin care active ingredient or (c2) at least one cellulose compound; and
(d) water.

2. The composition according to claim 1, wherein the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, provided that
the (i) fatty alcohols and the (ii) fatty acids comprise a branched alkyl group or a linear or branched alkenyl group, each having 4 to 30 carbon atoms,
the (iii) monovalent carboxylic acid monoesters comprise a branched alkyl group or a linear or branched alkenyl group, each having 4 to 30 carbon atoms,
the (iv) divalent carboxylic acid diesters comprise at least one branched alkyl group having 3 to 10 carbon atoms, and an alkylene group having 6 to 18 carbon atoms,
the (v) N-acyl amino acid esters comprise a branched alkyl group having 3 to 10 carbon atoms, and a linear or branched acyl group having 4 to 30 carbon atoms, and
the (vi) alkyl lactates comprise a linear or branched alkyl group or a linear or branched alkenyl group, each having 4 to 30 carbon atoms.

3. The composition according to claim 1, wherein the (a) oil is selected from (v) N-acyl amino acid esters comprising, at least, a branched alkyl group having 3 to 10 carbon atoms.

4. The composition according to claim 1, wherein the amount of the (a) oil(s) in the composition ranges from 0.5% to 25% by weight relative to the total weight of the composition.

5. The composition according to claim 1, wherein the (b) diol having at least one carbon chain comprising 3 or more carbon atoms is selected from the group consisting of propylene glycol, dipropylene glycol, polypropylene glycol, butylene glycol, dibutylene glycol, polybutylene glycol, pentylene glycol, dipentylene glycol, hexylene glycol, dihexylene glycol, and a mixture thereof.

6. The composition according to claim 1, wherein the weight ratio of the amount of the (b) diol having at least one carbon chain comprising 3 or more carbon atoms to the amount of the (a) oil is 4 or more.

7. The composition according to claim 1, wherein the (c1) skin care active ingredient has a Log P value of 4 or less.

8. The composition according to claim 1, wherein the (c1) skin care active ingredient is a skin care cosmetic active ingredient.

9. The composition according to claim 1, wherein the amount of the (c1) skin care active ingredient(s) in the composition ranges from 0.01% to 25% by weight relative to the total weight of the composition.

10. The composition according to claim 1, wherein the (c2) cellulose compound is selected from the group consisting of unmodified cellulose and nonionic or anionic cellulose ethers.

11. The composition according to claim 1, wherein the (c2) cellulose compound is selected from the group consisting of cellulose, cellulose gum, hydroxymethylcellulose, hydroxyethylcellulose, hydroxypropylmethylcellulose, sodium carboxymethylcellulose, and a mixture thereof.

12. The composition according to claim 1, wherein the amount of the (c2) cellulose compound(s) in the composition ranges from 0.01% to 40% by weight relative to the total weight of the composition.

13. The composition according to claim 1, wherein the composition has a turbidity of 120 NTU or less.

14. The composition according to claim 1, wherein the composition is a cosmetic composition.

15. A cosmetic process for treating a keratin substance, comprising:
applying the composition according to claim 1 to the keratin substance.
